(19) United States
(12) Patent Application Publication
(10) Pub. No.: US 2014/0379016 A1
(43) Pub. Date: Dec. 25, 2014

(54) LANCING DEVICE

(71) Applicant: Tianjin Huahong Technology Co., Ltd., Tianjin (CN)

(72) Inventor: Libo ZHANG, Tianjin (CN)

(21) Appl. No.: 14/314,052

(22) Filed: Jun. 25, 2014

(30) Foreign Application Priority Data
Jun. 25, 2013 (CN) 201320369954.0

(51) Int. Cl. A61B 5/151; A61B 5/15

(52) U.S. Cl. CPC A61B 5/1519; A61B 5/1411; A61B 5/15113; A61B 5/15194
USPC 606/182

(57) ABSTRACT

A lancing device, including a main body and a head detachably connected to the main body. The main body includes a main sleeve, a guide sleeve fastened and inserted in the main sleeve, an inner core slidably installed in the guide sleeve, a tail cap component connected with the rear end of the inner core, a feeding pressure spring and a withdrawing pressure spring which are sleeved on the inner core, and a shoot key installed on the main sleeve. The guide sleeve is uniformly provided with several spring plates folding towards the inner core at positions close to the front end. The inner side of the front end of each spring plate contacts the outer profile of the inner core. The inner core is equipped with a stopper. The stopper is located at the rear end of the guide sleeve.

### CROSS-REFERENCE TO RELATED APPLICATIONS

Pursuant to 35 U.S.C. §119 and the Paris Convention Treaty, this application claims the benefit of Chinese Patent Application No. 201320369954.0 filed Jun. 25, 2013, the contents of which, are incorporated herein by reference. Inquiries from the public to applicants or assignees concerning this document or the related applications should be directed to: Matthias Scholl P. C., Attn.: Dr. Matthias Scholl Esq., 245 First Street, 18^{th} Floor, Cambridge, Mass. 02142.

### BACKGROUND OF THE INVENTION

1. Field of the Invention

The invention relates to peripheral blood sampling instruments in the clinical medical field, and more particularly to a lancing device.

2. Description of the Related Art

Lancing devices are one of major peripheral blood sampling instruments. A lancing device consists of a main body and a head. The head is detachably connected with the front end of the main body. The main body comprises a main sleeve, a guide sleeve, an inner core, a tail cap component, a feeding pressure spring, a withdrawing pressure spring, a shoot key, etc. Some lancing devices are also provided with a push key. The guide sleeve is in close fit with and inserted in the main sleeve; the inner core is slidably installed in the guide sleeve; the tap cap component is stalled at the rear end of the main sleeve, connected with the inner core through the withdrawing pressure spring therein and a stopper installed at the rear end of the inner core; the feeding pressure spring is sleeved on the inner core, limited in the front and the back through a shoulder side arranged at the inner core and a partition located in the main sleeve; the shoot key is installed at the main sleeve, with a contact aligned with an elastic arm on the inner core; and the push key is slidably installed at the main sleeve, the guide sleeve and the inner core along the feeding direction. The peripheral blood sampling with the above lancing device usually has the following steps:

1: removing the head;

2: pulling the tail cap component to drive the inner core to move backward such that the feeding pressure spring is in the compressed state;

3: inserting a disposable blood-taking needle at the front end of the inner core;

4: installing the head at the main body;

5: moving the front end face of the head to contact the finger pulp, press the shoot key such that the spring arm swings and departs from a limiting hole on the guide sleeve; at this time, by the action of the feeding pressure spring, the inner core drives the disposable blood-taking needle to move forward, pass through the front end hole of the head and puncture the finger pulp; after needle tip reaches a certain depth, by the action of the withdrawing pressure spring, the inner core drives the disposable blood-taking needle to move back such that the needle tip of the disposable blood-taking needle returns back into the head, thus completing the entire blood-taking process;

6: removing the head again, directly pulling and discarding the used disposable blood-taking needle, or if the main body is provided with the push key, press the push key to eject the disposable blood-taking needle out.

The lancing device can make patients feel less pain in use. Overcoming the dread feeling of patients is one of aspects that should be continuously improved and perfected. The reduction of the pain feeling is mainly achieved through improving the needle feeding speed or needle feeding stability, etc. At present, the lancing device is required to be improved on both aspects.

### SUMMARY OF THE INVENTION

In view of the above-described problems, it is one objective of the invention to provide a lancing device which has minimum fit clearance between the inner core and the guide sleeve, can reduce needle jitter and improve stability of feeding and withdrawing the needle to ensure patient comfort.

To achieve the above objective, in accordance with one embodiment of the invention, there is provided a lancing device, comprising a main body and a head detachably connected to the main body. The main body comprises a main sleeve, a guide sleeve fastened and inserted in the main sleeve, an inner core slidably installed in the guide sleeve, a tail cap component connected with the rear end of the inner core, a feeding pressure spring and a withdrawing pressure spring which are sleeved on the inner core, and a shoot key installed on the main sleeve. The guide sleeve is uniformly provided with several spring plates folding towards the inner core at positions close to the front end; the inner side of the front end of each spring plate contacts the outer profile of the inner core; the inner core is equipped with a stopper; the stopper is located at the rear end of the guide sleeve; and when the feeding pressure spring is compressed, its front end face is pressed against the stopper.

In a class of this embodiment, the inner side of each spring plate is provided with a guide slot; the outer profile of the inner core is provided with a matched guide projection; and the guide projection is inserted in the guide slot.

In a class of this embodiment, the number of the spring plates is two, and the two spring plates are arranged at an angle of 180°.

The invention has the following advantages and beneficial effects:

The invention provides the above technical solution. The several spring plates envelop the inner core to reduce the fit clearance between the guide sleeve and the inner core such that the fit clearance between the two approaches to zero, thus reduce needle jitter; when the inner core drives the stopper to move to the rear end face of the guide sleeve, the stopper and the several spring plates form a front-and-rear dual support structure to ensure that the disposal blood-taking needle steadily punctures and exits from the finger pulp, thus improving the stability of feeding and withdrawing the needle. Due to reduction of the needle jitter and improvement on the stability of feeding and withdrawing the needle, patients feel less pain.

### BRIEF DESCRIPTION OF THE DRAWINGS

FIG. 1 is a schematic diagram of a lancing device in accordance with one embodiment of the invention;

FIG. 2 is a schematic diagram of a guide sleeve of a lancing device in FIG. 1;

FIG. 3 is an enlarged view of FIG. 1 at position III.

In the drawings, the following reference numbers are used: 1—Main body; 11—Main sleeve; 12—Guide sleeve; 121—Spring plate; 122—Guide slot; 13—Inner core; 131—Guide projection; 14—Tail cap component; 141—Tail cap; 142—Spacer sleeve of tail cap; 15—Feeding pressure spring; 16—Withdrawing pressure spring; 17—Shoot key; 18—Stopper; 2—Head; 21—Cap; 22—Inner core of cap; 23—Connection barrel.

### DETAILED DESCRIPTION OF THE EMBODIMENTS

For further illustrating the invention, experiments detailing a lancing device are described below. It should be noted that the following examples are intended to describe and not to limit the invention.

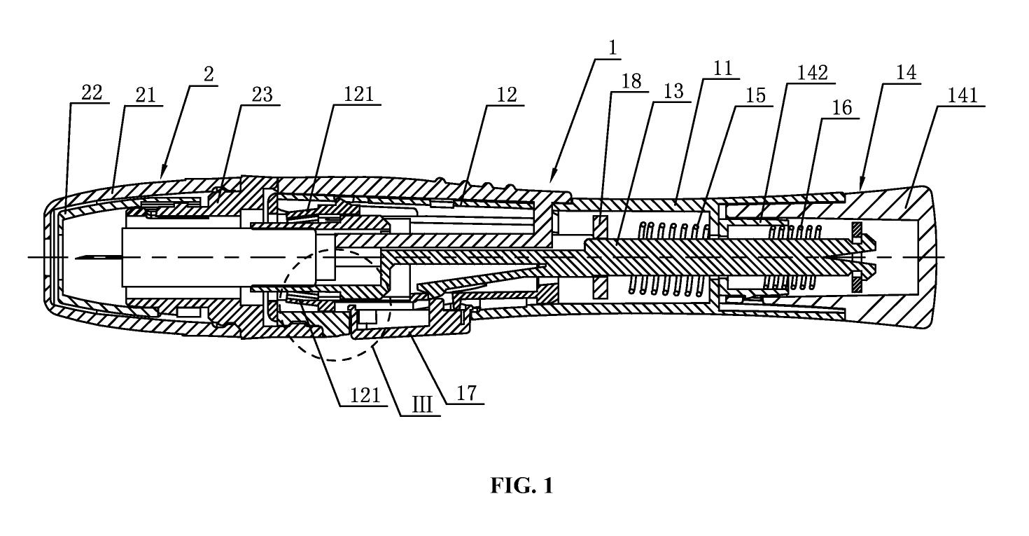

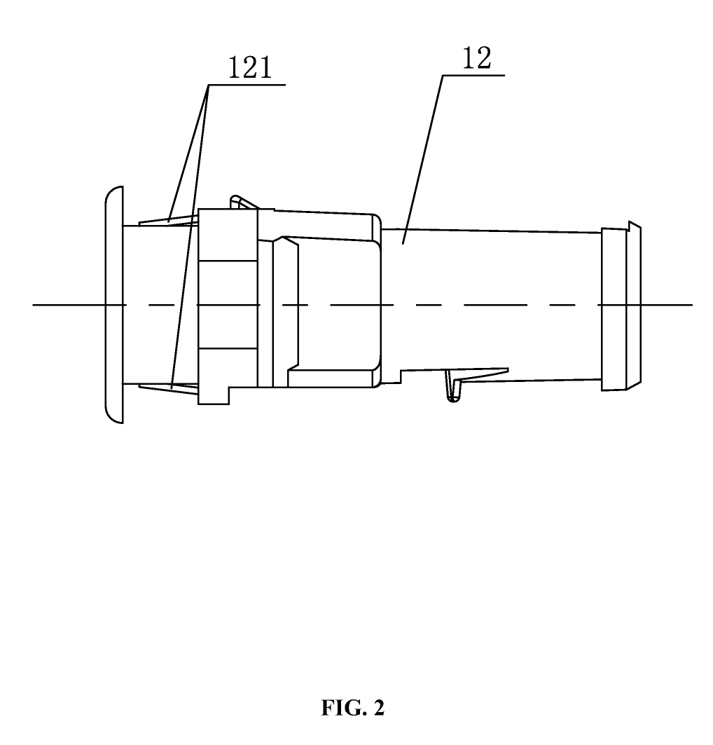

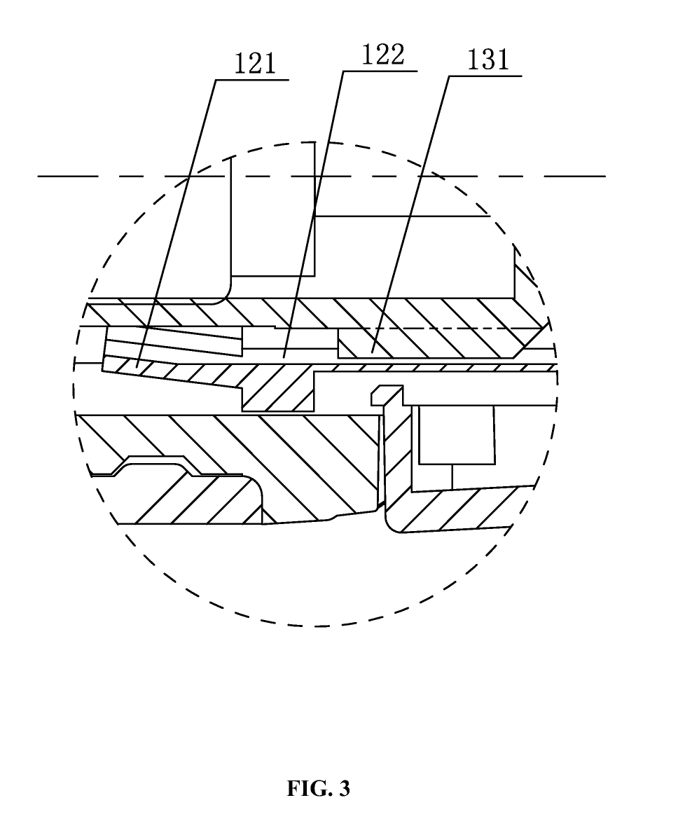

Refer to FIGS. 1-3, a lancing device, comprises a main body 1 and a detachably connected head 2. The structure of the head is identical with that of the existing lancing device, also comprising a cap 21, an inner core 22 of the cap and a connection barrel 23. Specifically, the rear end of the connection barrel is connected with the main body in spiral, pluggable, or other common detachable connection means. The main body comprises a main sleeve 11, a guide sleeve 12 fastened and inserted in the main sleeve, an inner core 13 slidably installed in the guide sleeve, a tail cap component 14 connected to the rear end of the inner core, a feeding pressure spring 15 and a withdrawing pressure spring 16 which are sleeved on the inner core, and a shoot key 17 installed on the main sleeve. The tail cap component is identical with that of the existing lancing device, also consisting of a tail cap 141 and a spacer sleeve 142 of the tail cap reversely installed and fixed in the tail cap. The withdrawing pressure spring is identical with the existing installation structure, installed in the tail cap component, limiting movement back and forth through the spacer sleeve of the tail cap and the stopper installed at the rear end of the inner core. The guide sleeve is uniformly provided with several spring plates 121 folding towards the inner core at positions close to the front end. The spring plates and the guide sleeve are integrally molded through injection. The inner side of the front end of each spring plate contacts the outer profile of the inner core. The inner core is equipped with a stopper 18. The stopper is located at the rear end of the guide sleeve. When the feeding pressure spring is compressed, its front end face is pressed against the stopper. Specifically, the stopper is slidably connected with the rear portion of the inner core. The shoulder side on the inner core for locating the feeding pressure spring limits the position of forward movement of the stopper.

In the above structure, the inner side of each spring plate is provided with a guide slot 122; the outer profile of the inner core is provided with a matched guide projection 131; and the guide projection is inserted in the guide slot. The fit length of the guide slot and the guide projection is determined upon the shooting path of the lancing device, meaning that the fit length of the two shall be at least greater than the shooting path of the lancing device, namely the entire path from the position the needle is pressed by the feeding pressure spring to the deepest position in the finger pulp where the needle punctures. The objective is to ensure that in the entire feeding process, the inner core always linearly moves without deviation, thus minimizing the pain feeling for patients.

In the above structure, the preferable number of the spring plates is two, and the two spring plates are arranged at an angle of 180°. The number and arrangement means of the spring plates satisfy the overall structure arrangement demand of the guide sleeve as much as possible and simply the structure on the premise of reducing the fit clearance between the guide sleeve and the inner core and reducing needle jitter.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A lancing device, comprising:
a) a main body, the main body comprising a main sleeve, a guide sleeve fastened and inserted in the main sleeve, an inner core slidably installed in the guide sleeve, a tail cap component connected with a rear end of the inner core, a feeding pressure spring and a withdrawing pressure spring which are sleeved on the inner core, and a shoot key installed at the main sleeve; and
b) a head detachably connected to the main body;
wherein
the guide sleeve is uniformly provided with several spring plates folding towards the inner core at positions close to the front end;
the inner side of the front end of each spring plate contacts an outer profile of the inner core;
the inner core is equipped with a stopper;
the stopper is located at a rear end of the guide sleeve; and
when the feeding pressure spring is compressed, its front end face is pressed against the stopper.

2. The lancing device of claim 1, wherein the inner side of each spring plate is provided with a guide slot; the outer profile of the inner core is provided with a matched guide projection; and the guide projection is inserted into the guide slot.

3. The lancing device of claim 1, wherein a number of the spring plates is two, and two spring plates are arranged at an angle of 180°.

4. The lancing device of claim 2, wherein a number of the spring plates is two, and two spring plates are arranged at an angle of 180°.
